(19) United States
(12) Patent Application Publication
(10) Pub. No.: US 2015/0344926 A1
(43) Pub. Date: Dec. 3, 2015

(54) Enzyme Hyaluronan-Lyase, Method of Production Thereof, Use Thereof and Method of Preparation of Low-Molecular Hyaluronan

(71) Applicant: CONTIPRO BIOTECH S.R.O., Dolní Dobrouc (CZ)

(72) Inventors: Lenka Knorova, Rakovnik (CZ); Dzianis Smirnou, Usti nad Orlici (CZ); Veronika Leierova, Letohrad (CZ); Martin Krcmar, Zamberk (CZ); Lukas Franke, Zamberk (CZ); Vladimir Velebny, Zamberk (CZ)

(73) Assignee: Contipro Biotech s.r.o.,, Dolní Dobrouc (CZ)

(21) Appl. No.: 14/430,731

(22) PCT Filed: Sep. 26, 2013
(86) PCT No.: PCT/CZ2013/000116

§ 371 Date: Mar. 24, 2015

(30) Foreign Application Priority Data
Sep. 27, 2012 (CZ) PV 2012-664

(51) Int. Cl. C12P 19/26; C12N 9/88

(52) U.S. Cl. CPC C12P 19/26; C12N 9/88; C12Y 402/02001

(57) ABSTRACT

The invention relates to an enzyme which is able to degrade hyaluronic acid and which is produced by fungi of the genus Fistulina (especially Fistulina hepatica). The degradation proceeds by lyase mechanism in which double bonds between C4 and C5 of glucuronic acid are formed. The invention also includes the process of preparation and purification of the enzyme and a possible practical use thereof for the preparation of low-molecular hyaluronan or of cosmetic or pharmaceutical devices. Further, the invention relates to the method of preparation of low-molecular hyaluronan.

### FIELD OF THE ART

The invention relates to an enzyme, optionally an enzyme preparation, which is capable of degradation of hyaluronic acid (hyaluronan). A method of production of the enzyme by means of cultivation of a fungus belonging to the genus Fistulina, the use thereof and a method of preparation of a low-molecular hyaluronan are disclosed as well.

### PRIOR ART

Hyaluronic acid is a linear polysaccharide consisting of disaccharidic units composed of glucuronic acid and N-acetylglucosamine. Having this structure, it ranks among glycosaminoglycans. It is found in extracellular matrix of soft connective tissues, where it exhibits a stabilizing and hydrating function. Nowadays, hyaluronic acid is most often obtained by biotechnological routes.

Hyaluronidases are enzymes which are capable of degrading hyaluronic acid or salts thereof to lower fragments. The term hyaluronidase is superior to the terms designating individual groups of enzymes having a degrading activity towards hyaluronan. According to the mechanism of the effect on the polysaccharide, hyaluronidases may be classified into several types. The first group includes mammalian enzymes (EC 3.2.1.35) which degrade β-1-4 bond of hyaluronic acid, giving rise to tetrasaccharides. The cleaving mechanism of these enzymes is hydrolytic. Another group is composed of hyaluronate-3-glycanohydrolase of leeches (EC 3.2.1.36) which form tetra and hexasaccharides. In this case hyaluronic acid is degraded hydrolytically as well. The third group includes bacterial hyaluronidases (EC 4.2.2.1). These enzymes are called hyaluronan-lyases and they degrade hyaluronic acid by the mechanism of β-elimination, giving rise to a double bond between C4 and C5 of glucuronic acid. Up to now, this type of cleaving (degradation) has been described for bacteria only. This patent application discloses hyaluronan-lyase of a new (non-bacterial) source.

The patent literature mentioned below relates to bacterial hyaluronan lyases. In all cases only a method of production of enzymes by fermentation is protected. Always bacteria of the genera Streptococcus or Streptomyces are used. All hyaluronidases are produced in an extracellular way. Applications are usually disclosed as a direct medicinal, cosmetic or pharmaceutic use of the enzymes in specific compositions. Most frequently, it is an adjuvant assisting the penetration of a drug into epidermis in topical application. Compared to hyaluronidases having the hydrolytic cleaving mechanism, lyases are often very specific towards hyaluronic acid.

CH 628088 relates to the preparation of several products produced by streptococci. One of these products is also hyaluronan lyase.

JP 63044883 relates to hyaluronan-lyase SD-678 produced by the species Streptococcus dysgalactiae. The enzyme has the optimal activity within the pH of 5.8-6.6 at the temperature of 37° C. Ions inhibiting the hyaluronidase activity include e.g. Fe²⁺ or Cu²⁺.

JP 62104579 discloses hyaluronan-lyase produced by the group of C-genus Streptococcus, to which also Streptococcus dysgalactiae belongs. The molecular weight of the enzyme is 80 kDa. Said enzyme is highly specific towards hyaluronic acid. The optimal activity was observed at the pH of 6-7 and the temperature of 35-45° C. Moreover, stability was observed at 40° C. at pH 6.0 for 15 minutes.

U.S. Pat. No. 3,728,223 relates to the production of hyaluronan-lyase by the species Streptomyces hyalurolyticus. This enzyme is able to selectively cleave hyaluronic acid. The optimal pH is 5.0 and the optimal temperature is 60° C. However, the activity is retained even after heating to 70° C.

U.S. Pat. No. 6,902,548 relates to the use of hyaluronan-lyase produced by the species Streptomyces hyalurolyticus in ophtalmology. Upon purification, the enzyme preparation does not contain proteases anymore which have been an obstacle for such an application until then.

U.S. Pat. No. 4,258,134 relates to hyaluronan-lyase BMP-8231. This enzyme is produced by Streptomyces koganiensis. It is hyaluronic acid-specific. The optimal pH is 4.0, however, the stability was observed within the pH of 4.0-11.3. The optimal temperature for cleaving is 60° C. Moreover, the enzyme is stabile with respect to proteases.

WO 2010/130810 discloses hyaluronidase produced by the species Streptomyces koganiensis ATCC 31394. The molecular weight is 21.6 kDa. Isoelectric point is within the range of 4.4-4.8. The enzyme activity is 40000 I.U./mg or higher. Besides the isolation and purification process, the invention discloses the use of the enzyme for the preparation of pharmaceutical compositions or analytical agents.

U.S. Pat. No. 6,719,986 protects a preparation containing hyaluronan lyase assisting in the penetration of drugs into the epidermis. The production of lyase is not disclosed in detail therein. Only the production species Streptococcus agalactiae is mentioned. The molecular weight of the enzyme is 116 kDa and the isoelectric point is 8.6.

US 2010/0172892 discloses hyaluronan-lyase produced by Streptomyces actinocidus 77. The structure of the enzyme is disclosed therein in great detail and is claimed by claims. The use of the enzyme for the preparation of compositions useful in cosmetics, medicine or pharmacy, preferably for topical application, is claimed. The optimal cleaving conditions are: pH 6.5-7.0 and the temperature of 50-60° C. The enzyme has the isoelectric point at pH 4.4 and the molecular weight of 44 kDa. It has a low activity, or none at all, towards chondroitin sulphate or heparin. The enzyme activity is inhibited by ions of iron and copper. The identical enzyme is disclosed in WO 2009/037566 as well.

The preparation of low-molecular hyaluronan by means of bacterial lyases (Streptomyces hyalurolyticus) is mentioned in the document U.S. Pat. No. 6,613,897. However, it is only the preparation of oligosaccharides before their further chemical modification.

Up to now, neither any specialist literature, nor any patent has disclosed hyaluronidases having an elimination (lyase) mechanism of cleaving, produced by other organisms than bacteria.

### SUBJECT-MATTER OF THE INVENTION

The invention relates to a new hyaluronidase produced by fungi which has lyase (elimination) cleaving mechanism. This enzyme may be produced by fungi of the genus Fistulina, especially Fistulina hepatica.

The presented invention further relates to the method of obtaining the enzyme and the possible use thereof. The method of cultivation of fungi and isolation of the enzyme is not strictly given. The essential parameter is the source of the enzyme, i.e. the fungi of the genus Fistulina, which leads to hyaluronan-lyase. One of the possible methods of production of the enzyme is e.g. submerged cultivation of fungi. The cultivation optimally proceeds at 20 to 30° C. for 5 to 11 days. The cultivation may be carried out both in shake Erlenmeyer flasks, and in a fermenter. The cultivation medium contains a source of carbon, such as saccharose, further a source of nitrogen, such as yeast autolysate, and inorganic salts, such as Na₂HPO₄.12 H₂O and MgSO₄.7H₂O. The enzyme may be isolated from the cultivation medium after removing the mycelium and/or by extraction from the mycelium after the disintegration thereof. After the centrifugation the enzyme is further purified by methods known in the art, e.g. by washing with a buffer having the pH of 7.0. Before the chromatography separation, the solution containing the enzyme is exchanged by a suitable buffer which is necessary for the optimal course thereof. The separation on anion exchange sorbents seemed to be the most suitable method. However, other types of chromatography may be used as well. In case of the requirement of higher purity of the preparation, gel permeation chromatography may be used.

The lyase produced by fungi is characterized by the activity in a broad range of pH (3.5-8.0), having the optimum at pH 4.0, and in a broad range of temperatures (5 to 50° C.), having the optimum at 20° C. The enzyme is stable at the cleaving conditions for up to several weeks. The activity of the enzyme may be increased by 10-30% by means of addition of MgSO₄, MnCl₂, KCl or CuSO₄, in an amount of 5 mM to 20 mM with respect to the solution of the acid. For other enzymes (JP 63044883, WO 2009/037566), Cu²⁺ often acts as an inhibitor.

The production of hyaluronan-lyase from Fistulina hepatica is advantageous because it is a wood-destroying fungus, the so-called brown rot fungi. This fungus produces neither any toxic metabolites, nor any endotoxins, which may be the case of bacteria. The enzyme may be used for the preparation of low-molecular hyaluronan or derivatives thereof. The molecular weight of the final products may be affected by the degradation conditions, such as the time, temperature, the concentration of the hyaluronic acid in the solution or the ratio of the enzyme to the acid. The degradation takes place in an aqueous solution within the pH of 3.5 to 8.0 and the temperature of 5 to 50° C. for 1 minute to 30 days. Preferably, the degradation proceeds at pH 4.0 and temperature 20° C. for 24 to 168 hours. The hyaluronic acid used may originate from various sources (cocscombs, Streptococcus zooepidermicus, Streptococcus equismilis) and it may have any molecular weight, such as within the range of 1.5 to 2.2 MDa, nevertheless, the term high-molecular HA means HA which has a weight average molecular weight of 0.8 MDa or higher. The solution of the acid may be prepared within the concentration from 0.1 to 10% by weight. Besides the native hyaluronic acid, also salts thereof may be used, such as Na or K, or derivatives thereof, such as acyl derivatives, and it is possible to thereby prepare functionally specific oligosaccharides or other low-molecular products.

Further, the prepared enzyme may also be used for the preparation of pharmaceutical or cosmetic compositions as an adjuvant assisting in the penetration of substances into tissues.

### DESCRIPTION OF THE DRAWINGS

FIG. 1 represents the cleaving mechanism of hyaluronic acid by means of hyaluronan-lyase according to the invention.

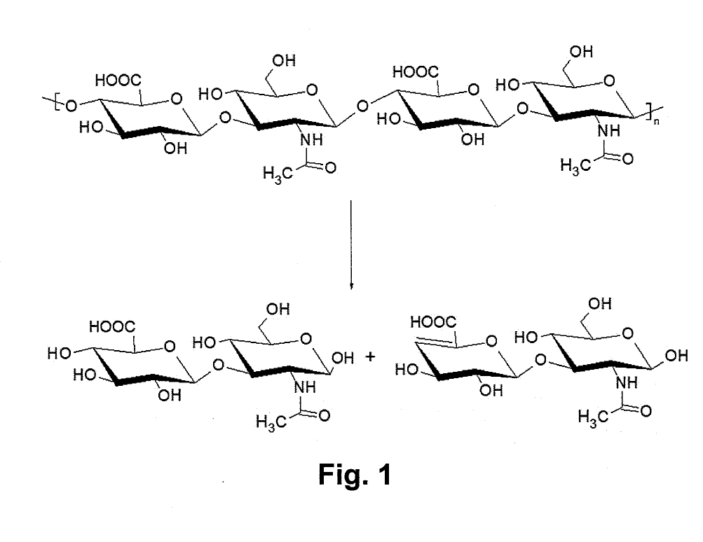

FIG. 2 represents chromatograms of the oligosaccharides formed by cleaving of hyaluronic acid. Detection at 210 and 232 nm.

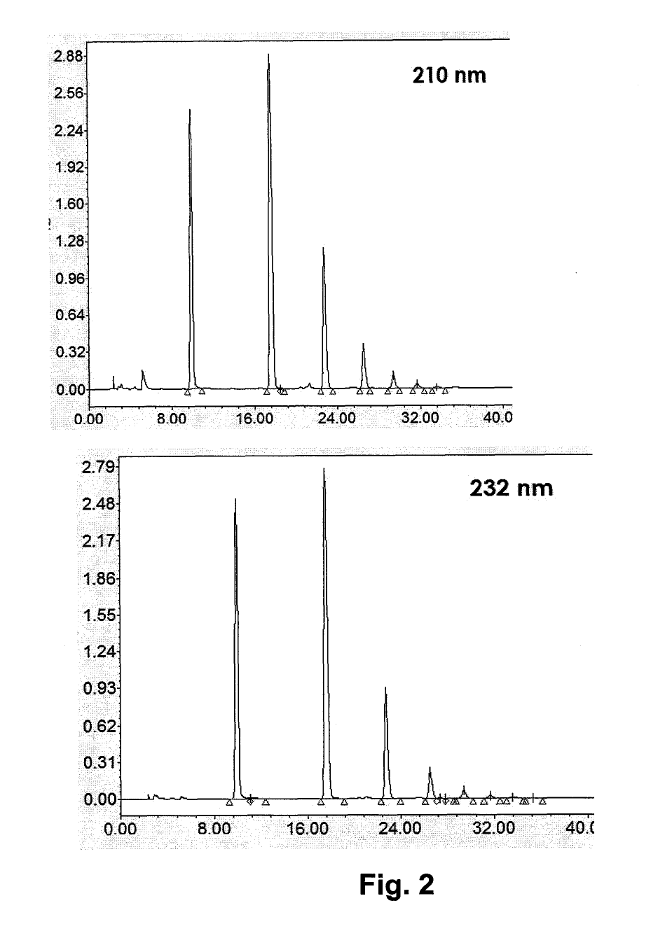

FIG. 3 represents the dependence of the relative enzyme activity on the pH, wherein the relative enzyme activity is the ratio of the enzyme activity at the given pH to the maximal enzyme activity, i.e. at the optimal pH of 4.0, ×100%.

FIG. 4 represents the dependence of the relative enzyme activity on the temperature, wherein the relative enzyme activity is the ratio of the enzyme activity at the given temperature to the maximal enzyme activity, i.e. at the optimal temperature of 20° C., ×100%.

FIG. 5 represents the increase of the enzyme activity after the addition of salts, wherein the dependence of the relative enzyme activity on the addition of a specific salt in a specific amount is plotted, wherein the relative enzyme activity is the ratio of the enzyme activity with the addition of the salt to the enzyme activity without the addition of the salt, ×100%.

### PREFERRED EMBODIMENTS OF THE INVENTION

The invention is explained, but not limited, by the following examples of the practical embodiments.

In case molecular weight (Mr) is mentioned throughout the description, e.g. that of hyaluronan, a weight average molecular weight is meant. The term “high-molecular” hyaluronan includes hyaluronan having Mr higher than 0.8 MDa. The term “low-molecular” hyaluronan includes hyaluronan having Mr within the range of approximately 0.3 to 500 kDa. The term “hyaluronan” includes both hyaluronic acid and salts thereof (such as Na or K).

### EXAMPLE 1

The cultivation of fungi (Fistulina hepatica) for the purpose of production of the enzyme or enzyme preparation took place in a liquid medium composed of 35 g/l of saccharose, 3 g/l of yeast autolysate, 2.5 g/l of Na₂HPO₄.12H₂O and 0.5 g/l of MgSO₄.7H₂O, wherein the stated amounts of the components are with respect to 1 l of the medium. The cultivation was conducted at 25° C. for 6 days. Erlenmeyer flasks having 1-l volume were used, containing 500 ml of the culture medium. For inoculation of the flasks, 1 culture-grown Petri dish of 9 cm-diameter for 1 l of the culture medium was used. The flasks were either used directly for the isolation of the enzyme or for inoculation of the fermenter.

After the cultivation, mycelium was removed from the medium, e.g. by means of centrifugation or filtration through a polyamide filter cloth. The enzyme may also be obtained from the mycelium, but in a substantially lower amount, and therefore, only the recovery of the enzyme from the culture medium is further disclosed. In case of recovery of the enzyme from the mycelium, disintegration of the mycelium, e.g. by drastic freezing and grinding in a mortar, by means of ultrasound or by means of other methods, is performed.

For purification purposes, the culture medium was exchanged for a buffer having pH of 7.0, in such a way that first the medium was filtrated off by means of a membrane and then the sample was washed with the buffer (2 l), e.g. phosphate buffer, at least once. Then the enzyme may be used immediately for further purification by means of chromatography separation. In case the enzyme is not further purified, herein it is called “enzyme preparation”. Such a crude enzyme preparation may be used for cleaving of hyaluronic acid as well.

### EXAMPLE 2

In order to achieve a higher purity, the enzyme preparation prepared according to the Example 1 was further separated by means of chromatography techniques. Preferably, anion-exchange chromatography was used. Elution was performed with the linear gradient of NaCl within the range of 0-1 mol/l. For the best purity, the fractions were further separated by means of gel permeation chromatography.

The amount of the produced enzyme after the chromatography was within the range of 600±200 μg/ml, which makes approximately 1 mg of the pure enzyme in 1 l of the culture medium.

### EXAMPLE 3

Cleaving of hyaluronic acid was carried out in 0.1M acetate buffer having pH of 4.0. For this purpose, 1% solution of high-molecular hyaluronic acid (2 MDa) was prepared. The substrate was prepared biotechnologically. 4 ml of the solution of high-molecular hyaluronic acid were mixed with 200 μl of the enzyme solution in 0.02 M phosphate buffer having pH of 7.0 after chromatography, the enzyme concentration being 600 μg/ml. The cleaving proceeded at 20° C. for 3 days. The resulting products were hyaluronan oligosaccharides, mostly a mixture of disaccharides to dodeca-saccharides having a double bond between C4 and C5 of the terminal glucuronic acid. The average molecular weight of the oligosaccharides determined by means of gel permeation chromatography was within the range of 0.3 to 300 kDa and the terminal glucuronic acids had a double bond between C4 and C5.

### EXAMPLE 4

The resulted low-molecular hyaluronic acid fragments were quantified by means of RP-HPLC. Detection was carried out by a UV detector at 210 and 232 nm. An analysis of the resulted products showed lyase mechanism of cleaving. It is known that the unsaturated bonds which are formed exhibit an increased absorbance at 232 nm. Further, it was proved that all acid that was introduced into the reaction mixture was degraded to oligosaccharides.

### EXAMPLE 5

Cleaving of the hyaluronic acid derivative was carried out in 0.1M acetate buffer having pH of 4.0. For this purpose, 1% solution of palmitoyl hyaluronan (2 MDa) was prepared. 4 ml of said solution were mixed with 200 μl of enzyme solution in 0.02 M phosphate buffer having pH of 7.0 after chromatography, the enzyme concentration being 600 μg/ml. The substrate was prepared by a chemical synthesis from biotechnologically produced hyaluronic acid. The cleaving proceeded at 20° C. for 3 days. The resulting products were acylated hyaluronan oligosaccharides, more specifically a mixture of disaccharides to dodeca-saccharides having a double bond between C4 and C5 of the terminal glucuronic acid. The average molecular weight of the oligosaccharides determined by means of gel permeation chromatography was again within the range of 0.3 to 300 kDa and the terminal glucuronic acids had a double bond between C4 and C5.

### EXAMPLE 6

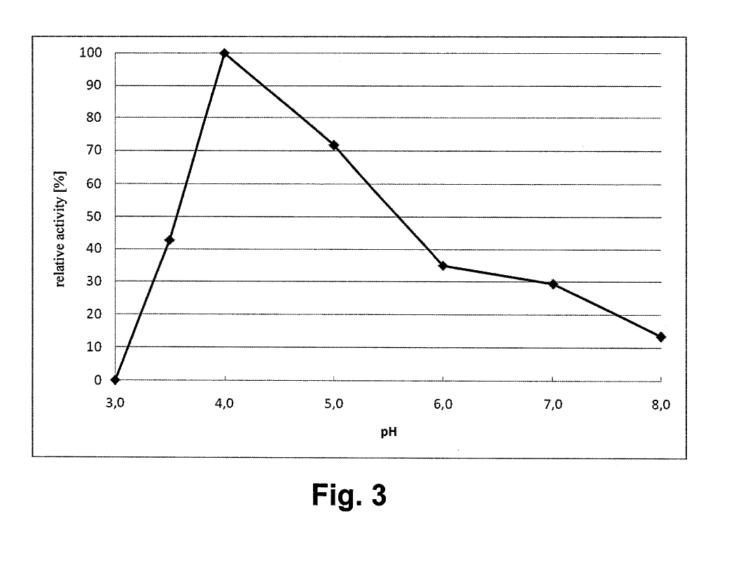

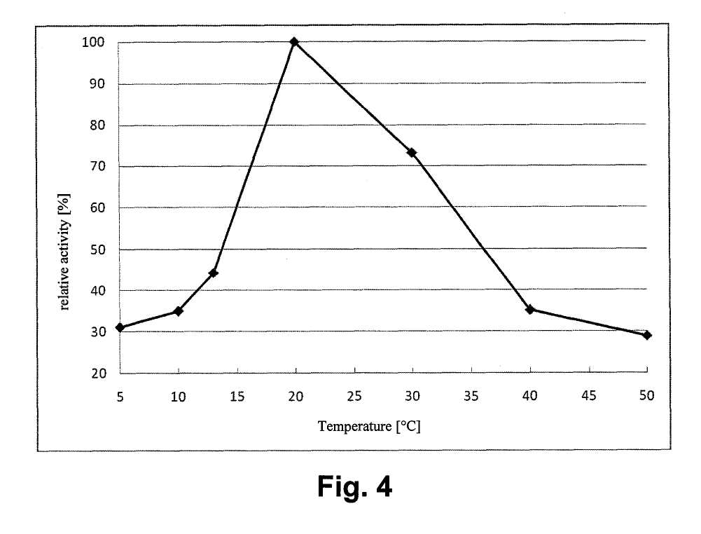

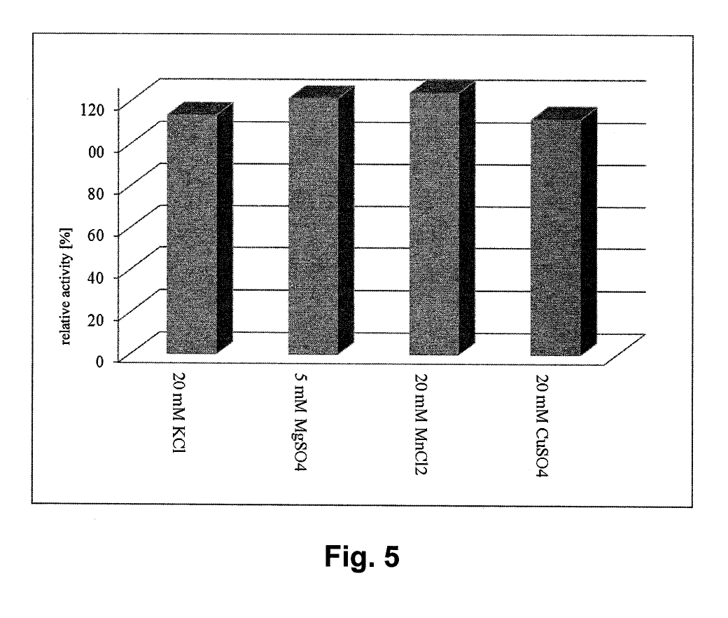

The enzyme activity depending on the temperature, on pH and on the added salts was observed by means of a rheometer. Decrease of the viscosity in time was monitored. For all experiments, 1% HA solution in the respective buffer (pH) was prepared, to which an enzyme solution after the chromatographic separation was pipetted (0.02M phosphate buffer, 600 μg/ml of enzyme). The volume of the hyaluronic acid solution was 460 μl and the volume of the enzyme was 40 μl. In case of testing the temperature dependence of the enzyme activity and determination of the optimal cleaving temperature, a buffer having pH of 4.0 was used. The pH dependence of the enzyme activity was tested at 37° C. Testing of the influence of salts was carried out by adding the respective salt into the hyaluronic acid solution. The detected results were plotted in graphs—see FIG. 3, FIG. 4 and FIG. 5. The graphs clearly show that the optimal temperature for cleaving is approximately 20° C., the optimal pH is approximately 4.0.

### EXAMPLE 7

Cleaving of hyaluronic acid was carried out in 0.1M acetate buffer having pH of 4.0, to which one of the salts was added up to the concentration of 20 mM. The salts were MgSO₄, MnCl₂, KCl, CuSO₄. For cleaving, 1% solution of high-molecular hyaluronic acid in such modified buffer was prepared. The substrate was prepared biotechnologically. 4 ml of high-molecular hyaluronic acid solution were mixed with 200 μl of enzyme solution in 0.02 M phosphate buffer having pH of 7.0 after chromatography, the enzyme concentration being 600 μg/ml. The cleaving proceeded at 20° C. for 3 days. The resulting products were hyaluronan oligosaccharides, mostly a mixture of disaccharides to dodeca-saccharides having a double bond between C4 and C5 of the terminal glucuronic acid. The average molecular weight of the oligosaccharides determined by means of gel permeation chromatography was within the range of 0.3 to 300 kDa and the terminal glucuronic acids had a double bond between C4 and C5.

1. A method of preparation of hyaluronan-lyase, characterized by that it is obtained by submerged cultivation of a fungus belonging to the genus Fistulina at the temperature of 20 to 30° C. for 5 to 11 days.

2. The method according to claim 1, characterized by that the fungus belonging to the genus Fistulina is the species Fistulina hepatica.

3. The method according to claim 1 or 2, characterized by that the culture medium contains a source of carbon, a source of nitrogen and inorganic salts.

4. The method according to claim 3, characterized by that the source of carbon is saccharose, the source of nitrogen is yeast autolysate and the inorganic salts are Na₂HPO₄.12 H₂O and MgSO₄.7H₂O.

5. The method according to any of claims 1 to 4, characterized by that the enzyme is isolated from the culture medium after removal of the mycelium and/or by extraction from the mycelium after the disintegration of the mycelium.

6. The method according to claim 5, characterized by that the enzyme is further washed at least once with a buffer having the pH of 7.0.

7. The method according to claim 6, characterized by that the enzyme is further purified by means of chromatographic separation.

8. Hyaluronan-lyase preparable by means of the method according to claim 1 by submerged cultivation of a fungus belonging to the genus Fistulina in a culture medium.

9. Hyaluronan-lyase according to claim 8, characterized by that the fungus of the genus Fistulina is Fistulina hepatica.

10. Hyaluronan-lyase according to claim 8 or 9, characterized by that it has the optimal activity at the pH of 4.0 and at the temperature of 20° C.

11. A use of the enzyme hyaluronan-lyase prepared by the method according to claim 1 by cultivation of a fungus belonging to the genus Fistulina for degradation of hyaluronan or a derivative thereof.

12. A use of the enzyme hyaluronan-lyase prepared by the method according to claim 1 by cultivation of a fungus belonging to the genus Fistulina for the preparation of pharmaceutical or cosmetic compositions as a substance assisting in the penetration of substances into tissues.

13. A method of preparation of low-molecular hyaluronan, characterized by that a 0.1 to 10% wt. aqueous solution of high-molecular hyaluronic acid, a salt thereof or a derivative thereof is prepared, having the pH within the range of 3.5 to 8.0, an aqueous solution of the enzyme hyaluronan-lyase prepared by the method according to claim 1 by cultivation of a fungus belonging to the genus Fistulina is added, and a reaction is let to proceed at the temperature of 5 to 50° C. for 1 minute to 30 days.

14. The method according to claim 13, characterized by that the reaction is let to proceed at pH of 4.0 and the temperature of 20° C. for 24 to 168 hours.

15. The method according to claim 13 or 14, characterized by that the high-molecular hyaluronic acid, a salt thereof or a derivative thereof has the molecular weight within the range of 1.5 to 2.2 MDa.

16. The method according to any of claims 13 to 15, characterized by that the salt of hyaluronic acid is a sodium or potassium salt and the derivative of hyaluronan is an acylated hyaluronan.

17. The method according to any of claims 13 to 16, characterized by that to a 0.1 to 10% wt. solution of high-molecular hyaluronic acid, a salt thereof or a derivative thereof, and of enzyme hyaluronan-lyase, 5 to 20 mM of a salt selected from the group including MgSO₄, MnCl₂, KCl, CuSO₄ are added.
